(19) United States
(12) Patent Application Publication
(10) Pub. No.: US 2018/0168802 A1
(43) Pub. Date: Jun. 21, 2018

(54) NASAL PROSTHESIS AND METHOD FOR MANUFACTURING THE SAME

(71) Applicant: SHENZHEN HAN'S 3D TECHNOLOGY CO., LTD., Shenzhen (CN)

(72) Inventors: Raymond WU, Walnut, CA (US); Quanxin SU, Shenzhen (CN)

(73) Assignee: SHENZHEN HAN'S 3D TECHNOLOGY CO., LTD., Shenzhen (CN)

(21) Appl. No.: 15/898,389

(22) Filed: Feb. 16, 2018

Related U.S. Application Data
(63) Continuation of application No. PCT/CN2016/093227, filed on Aug. 4, 2016.

(30) Foreign Application Priority Data
Aug. 19, 2015 (CN) 201510510508.0

(51) Int. Cl. A61F 2/18

(52) U.S. Cl. CPC A61F 2/186

(57) ABSTRACT

The present application discloses a nasal prosthesis and a method for manufacturing the same. The nasal prosthesis includes a nasal bridge element, and the nasal bridge element includes an attachment surface configured to be attached to a muscle tissue of a human nasal bridge and an outer surface configured to be attached to a skin tissue of the human nasal bridge. The nasal prosthesis further includes a fixing structure arranged on the nasal bridge element, the fixing structure is arranged on the nasal bridge element and is one selected from the group consisting of: a through-hole cluster and/or a column cluster, a through-hole cluster and a counterbore cluster, or a column cluster and a counterbore cluster. A top surface of the column cluster forms all or a part of the attachment surface or the outer surface at a side where the column cluster is arranged.

### CROSS-REFERENCE TO RELATED APPLICATIONS

This application is a continuation of International Patent Application No. PCT/CN2016/093227 with an international filing date of Aug. 4, 2016, designating the United States, now pending, and further claims priority benefit to Chinese Patent Application No. 201510510508.0 filed Aug. 19, 2015. The contents of the aforementioned applications, including any intervening amendments thereto, are incorporated in their entirety herein by reference.

### TECHNICAL FIELD

The present application relates to a nasal prosthesis for nasal shaping, and also relates to a method of making the nasal prosthesis.

### BACKGROUND

Cosmetic shaping of a nasal organ often requires use of a nasal prosthesis. As shown in FIG. 1, the nasal prosthesis 1 typically includes a structure of a nasal bridge element 2, a nasal tip element 3, and a nasal columella element 4 sequentially connected, and the nasal bridge element 2 includes an attachment surface 5 configured to be attached to a muscle tissue of the human nasal bridge and an outer surface 6 configured to be attached to a skin tissue of the human nasal bridge. As shown in FIG. 2, by cosmetic surgery, the nasal prosthesis 1 is implanted between the muscle tissue and the skin tissue at the nasal bridge element 8, the nasal tip element, and the nasal columella element, the appearance of the nasal bridge element 8 of a cosmetic surgery patient can be shaped by the nasal bridge element 2 of the nasal prosthesis, thereby achieving the cosmetic effect of changing the shape of the nasal bridge element of the cosmetic surgery patient.

Because nasal prosthesis materials for cosmetic surgery are produced in batches, according to a length dimension of human noses, external shapes and dimensions thereof are classified into several types for mass production. Thus, the nasal prosthesis material of the same type has the same external shape and dimension. During surgery, in order to modify the shape and the dimension of the nasal prosthesis material to fit with the shape and the dimension of the nose of the cosmetic surgery patient, generally, a cosmetic surgery doctor manually cuts and trims the nasal prosthesis according to the shape and the, dimension of the nose of the cosmetic surgery patient, to enable the shape and the dimension of the nasal prosthesis material to fit with the shape and the dimension of the nose of the cosmetic surgery patient as much as possible. Affected by the craft level, the surgery experience, and the mental status during the surgeon of the cosmetic surgery patient, the manual modification of the nasal prosthesis material makes a fitting degree between the nasal prosthesis of the shape and the dimension of the cosmetic surgery patient greatly varies, which affects the attachment between the nasal prosthesis and the nasal tissue, thereby make it difficult to ensure the surgeon quality.

In order to solve this problem, a method for engraving the nasal prosthesis material using a three-dimensional laser engraving technique has been proposed. The method includes acquiring data involving the shape and the dimension of the nasal bridge element of the cosmetic surgery patient, engraving the nasal bridge element of the nasal prosthesis material by a laser device based on the acquired data to acquire a nasal prosthesis with the shape and the dimension of the nasal bridge element highly fitting with the shape and the dimension of the nasal bridge element of the cosmetic surgery patient. Thus, such a method improves the fitting degree between the nasal prosthesis and the nasal bridge element of the cosmetic surgery patient.

However, the nasal prosthesis used in the prior art still has the following defects:

First, because in the nasal bridge element of the existing nasal prosthesis, the attachment surface configured to be attached to the muscle tissue of the human nasal bridge and the outer surface configured to be attached to the skin tissue of the human nasal bridge are both smooth, it is difficult for the nasal bridge element of the nasal prosthesis to be bonded with the muscle tissue and the skin tissue of the human nasal bridge after the surgery, which often results in movement of the nasal bridge element of the nasal prosthesis under the action of an external force, causing the deformation of the nasal shaping and seriously affecting the effect of the cosmetic surgery.

In addition, since the nasal prosthesis material often adopts a silicone rubber existing with transparency, when the nose of the cosmetic surgery patient after the surgery approaches a light source, the transparency of the nasal bridge element of the nasal prosthesis can be observed from a lateral side of the nose, thereby decreasing the anti-recognition of the cosmetic surgery effect.

### SUMMARY

One of technical problems to be solved by the present application is to provide a nasal prosthesis, which overcomes the defect that the existing nasal prosthesis is difficult to be tightly bonded with the muscle tissue and the skin tissue of the human nasal bridge and is prone to move, resulting in deformation of the nasal shaping.

A second technical problem to be solved by the present application is to provide a method for manufacturing the nasal prosthesis, which overcomes the defect that the existing nasal prosthesis is difficult to be tightly bonded with the muscle tissue and the skin tissue of the human nasal bridge and is prone to move, resulting in deformation of the nasal shaping.

In order to solve the first technical problem, the present application adopts the following technical solution: a nasal prosthesis is constructed. The nasal prosthesis comprises a nasal bridge element, the nasal bridge element comprising an attachment surface configured to be attached to a muscle tissue of a human nasal bridge and an outer surface configured to be attached to a skin tissue of the human nasal bridge; wherein, the nasal prosthesis further comprises a fixing structure arranged on the nasal bridge element, the fixing structure is arranged at the nasal bridge element and being one selected from the group consisting of a through-hole cluster and/or a column cluster, a through-hole cluster and a counterbore cluster, and a column cluster and a counterbore cluster, wherein a top surface of the column cluster forms all or a part of the attachment surface or the outer surface at a side where the column cluster is arranged.

In the nasal prosthesis of the present application, the fixing structure is a through-hole array extending from the attachment surface of the nasal bridge element to the outer surface of the nasal bridge element; or alternatively, the fixing structure is the through-hole array extending from the attachment surface of the nasal bridge element to the outer surface of the nasal bridge element and the counterbore array arranged at the attachment surface of the nasal bridge element, wherein the counterbore array and the through-hole array are spaced apart and alternated with one another.

to the nasal prosthesis of the present application, the fixing structure is one selected from the group consisting of a column array arranged at the attachment surface of the nasal bridge element; a column array arranged at the attachment surface of the nasal bridge element and the counterbore array arranged at the outer surface of the nasal bridge element; and a first column array and a second column array respectively arranged at the attachment surface of the nasal bridge element and the outer surface of the nasal bridge element.

In the nasal prosthesis of the present application, each first column of the first column array arranged at the attachment surface of the nasal bridge element has the same transverse dimension as each second column of the second column array arranged at the outer surface of the nasal bridge element, and a height of each first column is higher than a height of each second column; or each first column of the first column array arranged at the attachment surface of the nasal bridge element has the same height as each second column of the second column array arranged at the outer surface of the nasal bridge element and the transverse dimension of each first column is smaller than the transverse dimension of each second column; or the height of each first column of the first column array arranged at the attachment surface of the nasal bridge element is higher than that of each second column of the second column array arranged at the outer surface of the nasal bridge element, and the transverse dimension of each first column is smaller than the transverse dimension of each second column.

In the nasal prosthesis of the present application, the through-hole cluster and/or the column cluster, or the through-hole cluster and the counterbore cluster, or the column cluster and the counterbore cluster are distributed at the whole attachment surface or the whole outer surface of the nasal bridge element where the clusters are arranged; or alternatively, the through-hole cluster and/or the column cluster, or the through-hole cluster and the counterbore clusters, or the column cluster and the counterbore cluster are provided in the form of spaced sections along a length of the nasal bridge element on the attachment surface or the outer surface of the nasal bridge element where the clusters are arranged.

In the nasal prosthesis of the present application, the nasal bridge element is provided with a strip-shaped smooth region along an edge thereof; the through-hole cluster and/or the column cluster, or the through-hole cluster and the counterbore cluster, or the column cluster and the counterbore cluster are arranged within a region enclosed by the smooth region on the attachment surface or the outer surface of the nasal bridge element where the clusters are arranged; and the through-hole cluster and or the column cluster, or the through-hole cluster and the counterbore cluster, or the column cluster and the counterbore cluster are provided in the form of spaced sections along a length of the nasal bridge element within the region enclosed by the smooth region on the attachment surface or the outer surface of the nasal bridge element where the clusters are arranged.

In the nasal prosthesis of the present application, the nasal prosthesis further comprises a nasal tip element connected with the nasal bridge element; or further comprises the nasal tip element and a nasal columella element, wherein the nasal bridge element, the nasal tip element, and the nasal columella element are sequentially connected to form a shape of a human nose.

In order to solve the first technical problem, the present application adopts the following technical solution; a method for manufacturing a nasal prosthesis is provided. The nasal prosthesis comprises a nasal bridge element, and the nasal bridge element comprising an attachment surface configured to be attached to a muscle tissue of a human nasal bridge and an outer surface configured to be attached to a skin tissue of the human nasal bridge; wherein the method comprises; processing the nasal bridge element by a laser to form a fixing, structure arranged on the nasal bridge element and being one selected from the group consisting of a through-hole cluster and/or a column cluster, a through-hole cluster and a counterbore cluster, and a column cluster and a counterbore cluster, and atop surface of the column cluster forms all or a part of the attachment surface or the outer surface at a side where the column cluster is arranged; and the processing by the laser comprises the following steps:

S1: dividing a region to be processed of the nasal bridge element of the nasal prosthesis into a processing layer;

S2: adjusting a focus of the laser on a processing layer of a through-hole cluster to be processed, a counterbore cluster to be processed, or a column cluster to be processed;

S3: performing processing at a position of the through-hole cluster to be processed, the counterbore cluster to be processed, or the column cluster to be processed on the current processing layer; and

S4: finishing the processing if the current processing layer is a last layer, or otherwise, adjusting the focus of the laser to a next processing layer, and then return to S3.

In the nasal prosthesis of the present application, the number of the processing layer is one, two, three, or more.

The nasal prosthesis and the method for manufacturing the same of the present application, compared with the prior art, have the following advantages:

1. Because the fixing structure is arranged on the nasal bridge element of the nasal prosthesis, the nasal bridge element of the nasal prosthesis is configured to be attached to the muscle tissue of the human nasal bridge and the skin tissue of the human nasal bridge after surgery. After a postoperative recovery period, the muscle tissue and the skin tissue fill concave parts of the fixing structure, such that the nasal bridge element of the nasal prosthesis are embedded into the muscle tissue and the skin tissue of the human nasal bridge, the bonding is firm, thereby overcoming the defect that the nasal bridge element of the nasal prosthesis with the smooth surface is difficult to be tightly bonded with the muscle tissue and the skin tissue of the human nasal bridge and is prone to move, resulting in deformation of the nasal shaping.

2. Structures including the through-holes, the counterbores, and the column clusters are arranged on the nasal bridge element of the nasal prosthesis, which changes a transmission path of light in the nasal bridge element of the nasal prosthesis and enables the light passing through the nasal bridge element of the nasal prosthesis to diffuse, thereby reducing a transparency of the nasal bridge element of the nasal prosthesis and improving anti-recognition of the cosmetic effect.

3. The fixing structure uses a through-hole structure that allows body fluid secreted by tissues of the human nasal bridge to flow easily into the holes, such that the muscle tissue and the skin tissue of the human nasal bridge are more prone to grow, and the nasal bridge element of the nasal prosthesis are more tightly bonded to the muscle tissue and the skin tissue of the human nasal bridge of the nasal prosthesis.

4. It is ensured by the fixing structure that the attachment surface of the nasal bridge element is soft and can therefore better bonded with the muscle tissue of the human nasal bridge, meanwhile, it can also be ensured that the hardness of the attachment surface of the nasal bridge element is low and the hardness of the outer surface of the nasal bridge element is high, thereby being conducive to the attachment of the nasal bridge element of the nasal prosthesis to the muscle tissue of the nasal bridge element while ensuring the hardness of the outer surface of the nasal bridge element.

5. The strip-shaped smooth region is arranged on the nasal bridge element of the nasal prosthesis along the edge thereof, which can ensure that the edge of the nasal bridge element of the nasal prosthesis would not be embedded with the muscle tissue of the nasal bridge element, facilitating removal of the nasal prosthesis when necessary.

### BRIEF DESCRIPTION OF THE DRAWINGS

FIG. 1 is a lateral view of a common structure of a nasal prosthesis;

FIG. 2 is a schematic view of adopting a nasal prosthesis for nasal cosmetic surgery.

FIG. 3 is a first embodiment of a basic structure of the nasal prosthesis of the present application;

FIG. 4 is a second embodiment of the basic structure of the nasal prosthesis of the present application;

FIG. 5 is a third embodiment of the basic structure of the nasal prosthesis of the present application;

FIG. 6 is a front view of the second embodiment of the nasal prosthesis of the present application;

FIG. 7 is a front view of the third embodiment of the nasal prosthesis of the present application;

FIG. 8 is a front view of the fourth embodiment of the nasal prosthesis of the present application;

FIG. 9 is a front view of the fifth embodiment of the nasal prosthesis of the present application;

FIG. 10 is a top view of an attachment surface of the nasal bridge element of the present application with through-holes provided at the whole attachment surface;

FIG. 11 is a top view of the attachment surface of the nasal bridge element of the present application with the through-hole array provided in the form of spaced sections along a length of the nasal bridge element;

FIG. 12 is a top view of the attachment surface of the nasal bridge element of the present application with a narrow strip-shaped smooth region provided at a peripheral edge and with through holes provided within a region enclosed by the smooth region;

FIG. 13 is a top view of the attachment surface of the nasal bridge element of the present application with the narrow strip-shaped smooth region provided at the peripheral edge and with a through-hole array provided in the form of spaced sections along a length of the nasal bridge element within the region enclosed by smooth region; and

FIG. 14 is a flow chart of a method for manufacturing the nasal prosthesis of the present application.

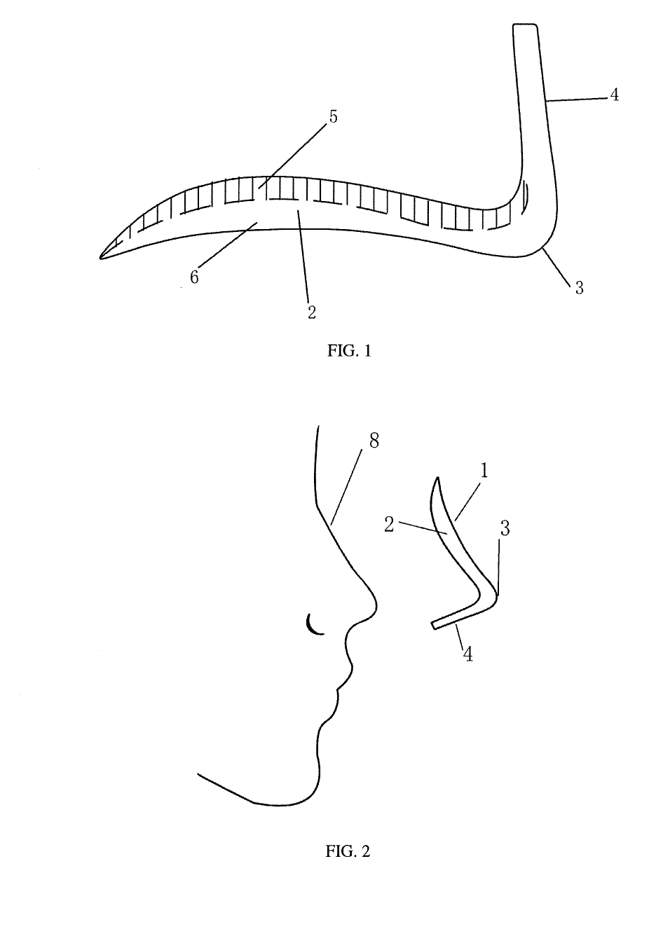

### DETAILED DESCRIPTION OF THE EMBODIMENTS

The present application is further described combining with the accompany drawings and embodiments.

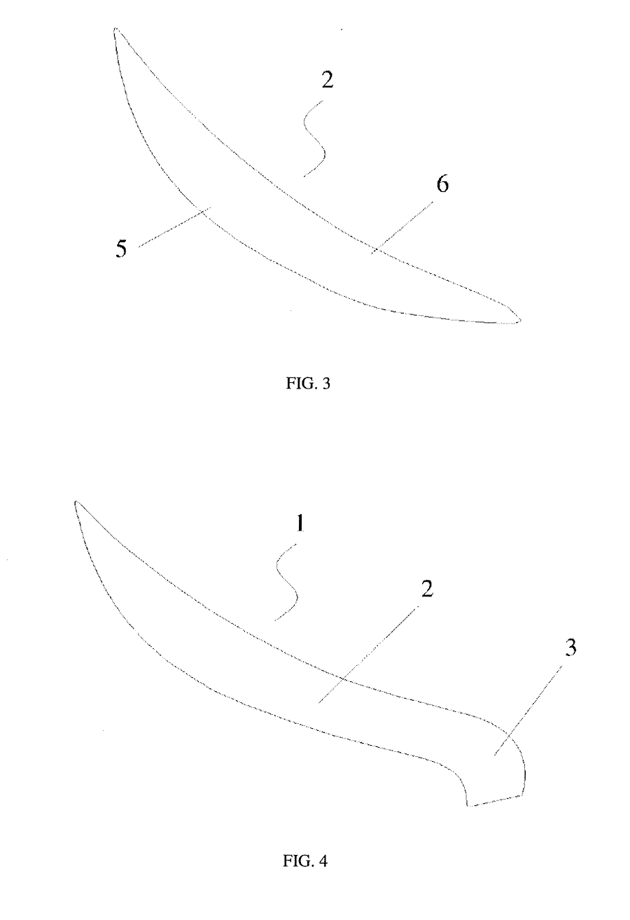

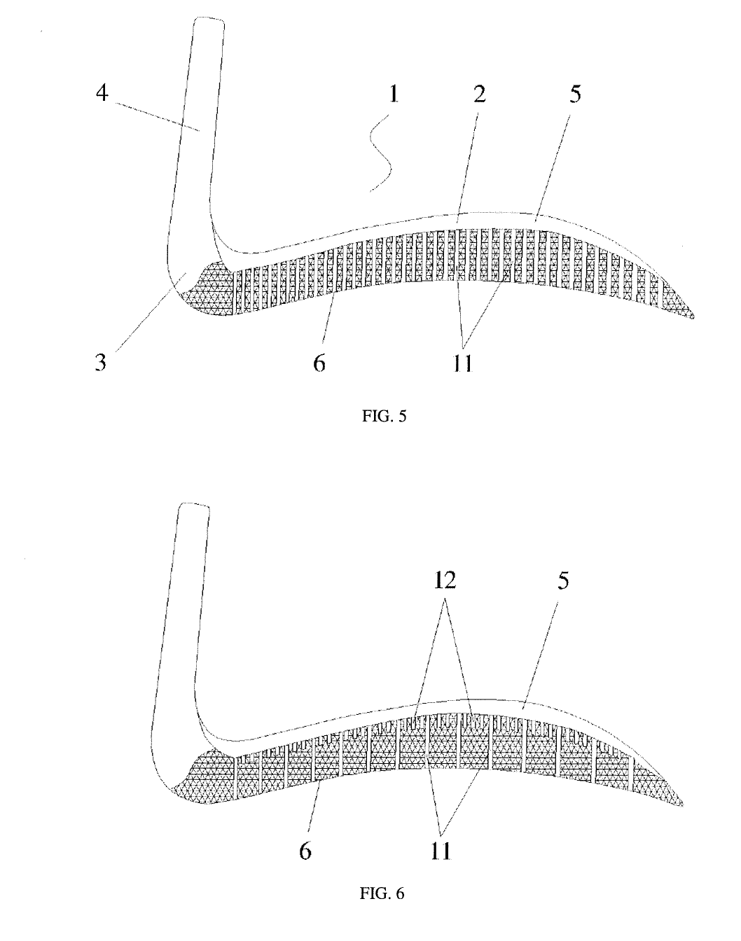

A nasal prosthesis of the present application may adopt three structure forms as show in FIG. 3-5. As shown in FIG. 3, the nasal prosthesis of the present application may only have the part of the nasal bridge element 2, the nasal bridge element comprises an attachment surface 5 configured to be attached to a muscle tissue of a human nasal bridge and an outer surface 6 configured to be attached to a skin tissue of the human nasal bridge. As shown in FIG. 4, the nasal prosthesis of the present application may consist of the nasal bridge element 2 and the nasal tip element 3, and the nasal tip element 3 is in connection with an end part of the nasal bridge element 2. As shown in FIG. 5, the nasal prosthesis 1 of the present application may also consist of the nasal bridge element 2, the nasal tip element 3, and a nasal columella element 4, and the nasal bridge element 2, the nasal tip element 3, and the nasal columella element 4 are sequentially connected to form a shape of a human nose.

The nasal bridge element of the nasal prosthesis of the present application is provided with a fixing structure, and the fixing structure adopts, but is not limited to the following structure so as to realize tight bonding between the nasal prosthesis and the human nasal bridge after the surgery:

1. a through-hole cluster,

2. a column cluster;

3. the through-hole cluster and the column cluster;

4. the through-hole cluster and a counterbore cluster; or

5. the column cluster and the counterbore cluster.

A top surface of the column cluster forms all or a part of the attachment surface or the outer surface at a side where the column cluster is arranged.

The structure of the nasal prosthesis of the present application is described taken the nasal prosthesis consisting of the nasal bridge element, the nasal tip element, and the nasal columella element as an example, the structure of the nasal bridge element of the following examples are also applicable to the nasal prosthesis adopting the structure formed by only the nasal bridge element and the nasal prosthesis adopting the structure consisting of the nasal bridge element and the nasal tip element.

### Embodiment 1

As shown in FIG. 5, the nasal prosthesis 1 of the present application comprises: the nasal bridge element 2, the nasal tip element 3, and the nasal columella element 4. The nasal bridge element 2 is provided with the fixing structure. The fixing structure is a through-hole array 11 extending from the attachment surface 5 of the nasal bridge element to the outer surface 6 of the nasal bridge element.

As shown in FIG. 10, in this embodiment, the through-hole array 11 is arranged on the whole attachment surface 5. Such arrangement of the through-hole array 11 has the flowing benefits: the whole attachment surface 5 and the whole outer surface 6 of the nasal bridge element can be well bonded to the muscle tissue of the human nasal bridge and the skin tissue of the human nasal bridge respectively. However, incomplete holes with sharp edges exist on a peripheral edge of the nasal bridge element, and this part of structure will be embedded into the muscle tissue of the human nasal bridge and the skin tissue of the human nasal bridge, the bonds of which are particularly firm, when a cosmetic surgery patient needs to replace the prosthesis and re-shaping the nose, the removal of the nasal prosthesis is relatively difficult and increases the pain of the patient; in addition, the arrangement of the through-hole array 11 on the whole attachment surface 5 makes the hardness of the nasal bridge element of the nasal prosthesis 2 be relatively low, which cannot satisfy the relatively high hardness of the nasal bridge element of the nasal prosthesis expected by the cosmetic surgery patient.

Thus, as shown in FIG. 12, the nasal bridge element 2 is provided with a strip-shaped smooth region 14 along an edge thereof, and a region enclosed by the smooth region 14 is provided with the through-hole array 11. In this way, the edge of the nasal bridge element 2 adopting the strip-shaped smooth region 14 is smooth and would not be embedded in the muscle tissue of the human nasal bridge and the skin tissue of the human nasal bridge, and it is easy to take out the nasal prosthesis when it is required to replace the nasal prosthesis.

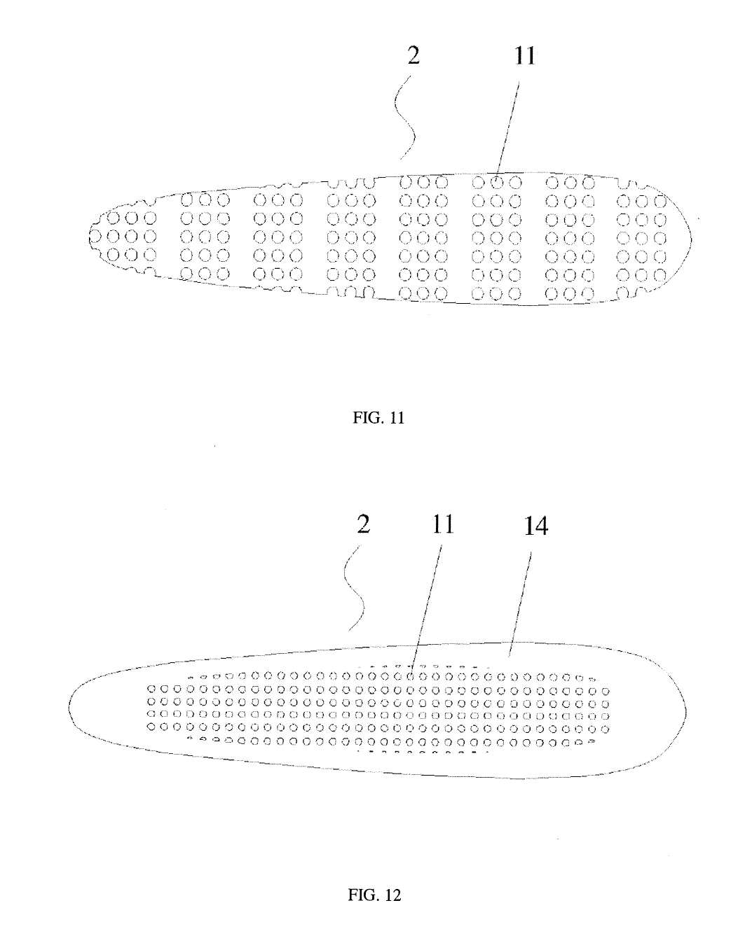

In order to solve the relatively low hardness of the nasal bridge element of the nasal prosthesis 2, as shown in FIG. 11, the through-hole array 11 is provided in the form of spaced sections along a length of the nasal bridge element on the attachment surface of the nasal bridge element, no through holes 11 is provided within spacing regions. Such structure can improve the hardness of the nasal bridge element of the nasal prosthesis 2.

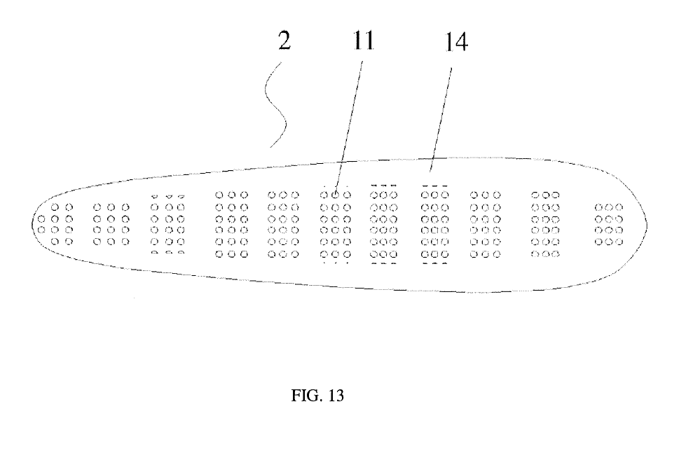

As shown in FIG. 13, a strip-shaped smooth region 14 is provided on the nasal bridge element of the nasal prosthesis 2 arranged along an edge thereof, the through-hole array 11 is provided in the form of spaced sections within a region enclosed by the smooth region 14, such that the problem of the embedding of the nasal bridge element of the nasal prosthesis into the muscle tissue of the human nasal bridge and the skin tissue of the human nasal bridge as well as the problem of the relatively low hardness of the nasal bridge element of the nasal prosthesis 2 can be solved.

The through-hole array 11 of the present embodiment can be replaced by adopting through-hole clusters of other structure. For example, the through-hole cluster which is symmetrically distributed along a symmetric center line of the nasal bridge element or the through-hole cluster which is arranged at an end part, a middle part, and in the proximity of a nasal tip element of the nasal bridge element can be adopted to replace the through-hole array 11.

### Embodiment 2

As shown in FIG. 6, a fixing structure on the nasal bridge element of the nasal prosthesis of this embodiment includes: a through-hole array 11 extending from the attachment surface 5 of the nasal bridge element to the outer surface 6 of the nasal bridge element, and a counterbore array 12 provided at the attachment surface 5 of the nasal bridge element, wherein the counterbore array 12 and the through-hole array 11 are spaced apart and alternated with one another.

The present embodiment adopts the arrangement of the counterbore array 12 on the attachment surface 5 of the nasal bridge element, on the one hand, the hardness of the attachment surface of the nasal bridge element 5 is reduced, such that it is ensured that the attachment surface of the nasal bridge element 5 and the muscle tissue of the human nasal bridge are well bonded and meanwhile the outer surface of the nasal bridge element has higher hardness than the attachment surface, thereby satisfying the shaping requirement; on the other hand, it is ensured that the bonding between the attachment surface 5 of the nasal bridge element and the muscle tissue of the human nasal bridge are much firmer.

The through-hole array 11 and the counterbore array 12 of the present embodiment may also adopt structures as shown in FIGS. 10-13. The through-hole array 11 and the counterbore array 12 may also be replaced by the through-hole cluster and the counterbore cluster of other structures. For example, the through-hole cluster and the counterbore cluster which are symmetrically distributed along a symmetric center line of the nasal bridge element, or the through-hole cluster and the counterbore cluster which are arranged at an end part, a middle part, and a proximity of a nasal tip element of the nasal bridge element can be adopted to replace the through-hole array 11 and the counterbore array 12.

The counterbore array 12 of this embodiment can be replaced by a structure of a column array in other embodiment, which can also achieve the object of the present application.

The counterbore array 12 of this embodiment can be arranged on the outer surface of the nasal bridge element in other embodiment, which can also achieve a basic object of the present application.

### Embodiment 3

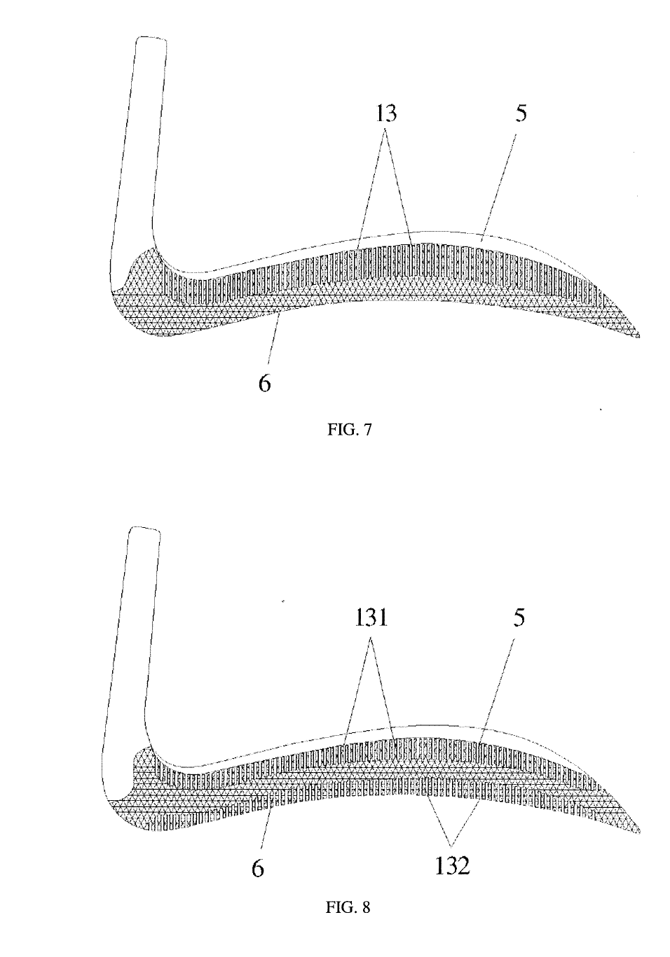

As shown in FIG. 7, a fixing structure on the nasal bridge element of the nasal prosthesis of this embodiment includes: a column array 13 arranged on the attachment surface 5 of the nasal bridge element, wherein a top surface of the column array 13 forms a part of the attachment surface 5 on which the column array 13 is arranged.

Because the column array 13 consists of multiple independent columns and an end part of each column is free end that can be easily deformed, the strength and the hardness of each column may be adjusted by changing the transverse dimension and the height of each column, and the shape of the cross section of each column may adopt circular shape, rectangular shape, or multilateral shape, etc. The fixing structure adopts the structure of the arrangement of the column array 13 on the attachment surface of the nasal bridge element 5, such that a relatively soft attachment surface of the nasal bridge element is acquired, and it is ensured that the attachment surface of the nasal bridge element 5 and the muscle tissue of the human nasal bridge are well bonded and meanwhile the outer surface of the nasal bridge element has higher hardness than the attachment surface, thereby satisfying the shaping requirement.

The column array 13 of the present embodiment may also adopt structures as shown in FIGS. 10-13. The column array 13 may also be replaced by the column cluster of other structures. For example, the column cluster which is symmetrically distributed along a symmetric center line of the nasal bridge element, or the column cluster which is arranged at an end part, a middle part, and in the proximity of a nasal tip element of the nasal bridge element can be adopted to replace the column array 13.

### Embodiment 4

As shown in FIG. 8, the fixing structure on the nasal bridge element of the nasal prosthesis of this embodiment includes: a first column array 131 and a second column array 132 respectively provided at the attachment surface of the nasal bridge element 5 and the outer surface 6 of the nasal bridge element. The strength and hardness of each column can be adjusted by changing the transverse dimension and the height of the column. For achieving the purpose that the hardness of the attachment surface 5 of the nasal bridge element is lower than the hardness of the outer surface 6 of the nasal bridge element, the following arrangement can be adopted:

1. It is ensured that each first column of the first column array arranged at the attachment surface 5 of the nasal bridge element has the same transverse dimension as each second column of the second column array arranged at the outer surface 6 of the nasal bridge element, and a height of each first column is higher than a height of each second column.

2. It is ensured that each first column of the first column array arranged at the attachment surface 5 of the nasal bridge element has the same height as each second column of the second column array arranged at the outer surface 6 of the nasal bridge element, and the transverse dimension of each first column is smaller than the transverse dimension of each second column.

3. It is ensured that the height of each first column of the first column array arranged at the attachment surface 5 of the nasal bridge element is higher than that of each second column of the second column array arranged at the outer surface 6 of the nasal bridge element, and the transverse dimension of each first column is smaller than the transverse dimension of each second column.

The first and second column arrays 131, 132 of the present embodiment may also adopt structures as shown in FIGS. 10-13. The first and second column arrays 131, 132 may also be replaced by column clusters of other structures. For example, the column clusters which are symmetrically distributed along a symmetric center line of the nasal bridge element, or the column clusters which are arranged at an end part, a middle part, and in the proximity of a nasal tip element of the nasal bridge element can be adopted to replace the first and second column arrays 131, 132.

### Embodiment 5

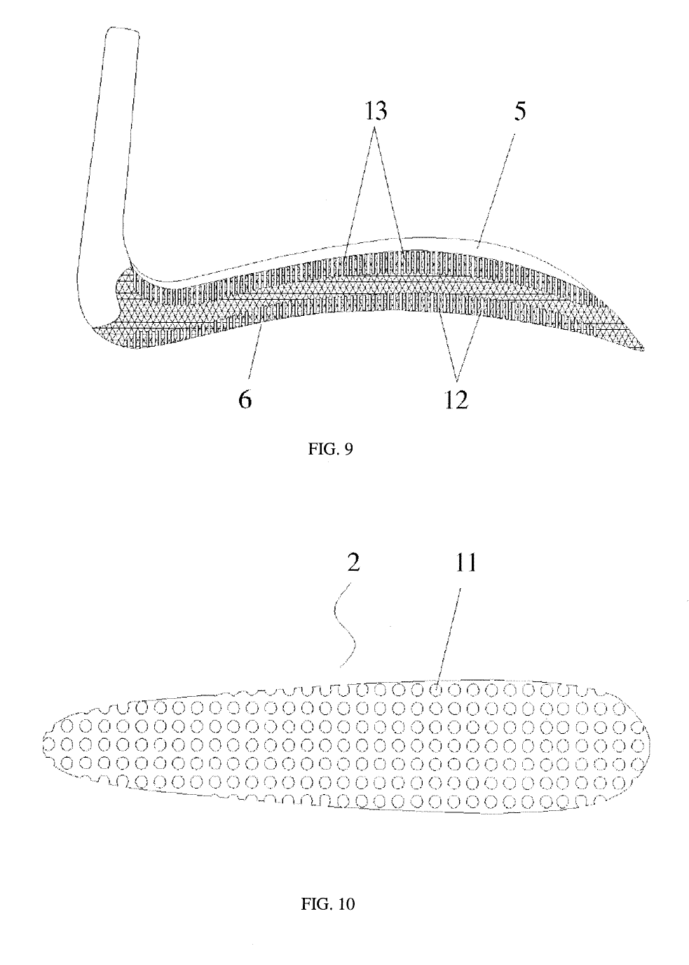

As shown in FIG. 9, the fixing structure on the nasal bridge element of the nasal prosthesis of this embodiment includes: a column array 13 arranged at the attachment surface of the nasal bridge element 5, and a counterbore array 12 arranged at the outer surface 6 of the nasal bridge element.

By adopting such a fixing structure, it can be ensured that the hardness of the attachment surface of the nasal bridge element is smaller than the hardness of the outer surface of the nasal bridge element.

The column array 13 and the counterbore array 12 of the present embodiment may also adopt structures as shown in FIGS. 10-13. The column arrays 13 and the counterbore array 12 may also be replaced by the column cluster and the counterbore cluster of other structures. For example, the column cluster and the counterbore cluster which are symmetrically distributed along a symmetric center line of the nasal bridge element, or the column cluster and the counterbore cluster which are arranged at an end part, a middle part, and a proximity of a nasal tip element of the nasal bridge element can be adopted to replace the column arrays 13 and the counterbore array 12.

In other embodiments, the fixing structure on the nasal bridge element of the nasal prosthesis adopts the structure including the counterbore array 12 arranged at the attachment surface of the nasal bridge element 5 and the column array 13 arranged at the outer surface 6 of the nasal bridge element, a basic object of the present application may also be achieved.

A method for manufacturing the nasal prosthesis of the present application adopts a laser to process the fixing structure arranged on the nasal bridge element, that is, a structure of the through-hole cluster and/or the column cluster, or a structure of the through-hole cluster and the counterbore cluster, or a structure of the column cluster and the counterbore cluster, and a top surface of the column cluster forms all or a part of the attachment surface or the outer surface at the side where the column cluster is arranged.

The nasal prosthesis manufactured by the manufacturing method can be the nasal bridge element, or a combination of the nasal bridge element and the nasal tip element, or a combination of the nasal bridge element, the nasal tip element, and the nasal columella element.

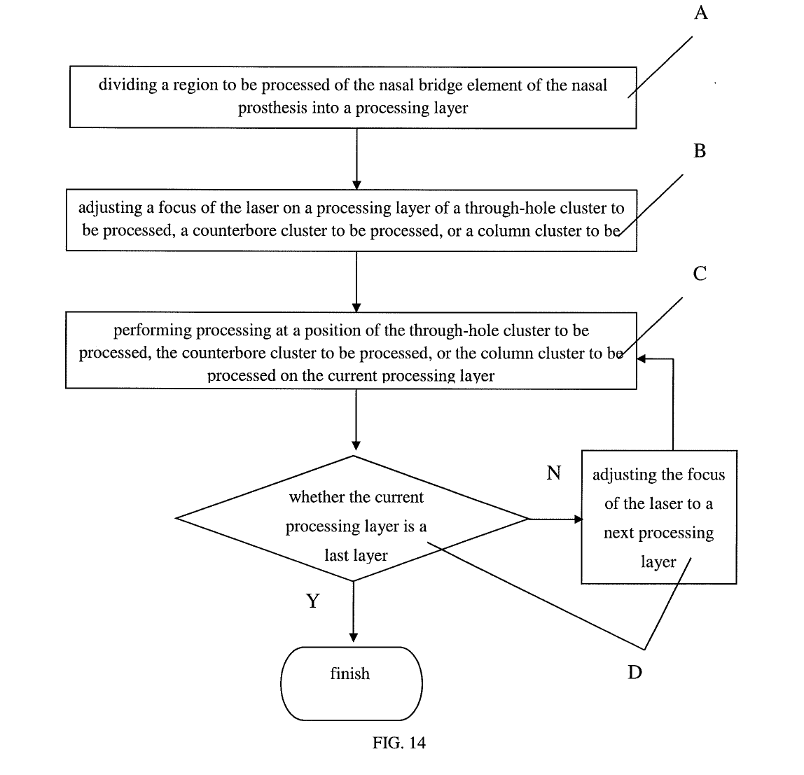

As shown in FIG. 14, in the method for manufacturing the nasal prosthesis, the processing by the laser comprises the following steps;

Step A: dividing a region to be processed of the nasal bridge element of the nasal prosthesis into a processing layer;

Step B: adjusting a focus of the laser on a processing layer of a through-hole cluster to be processed, a counterbore cluster to be processed, or a column cluster to be processed;

Step C: performing processing at a position of the through-hole cluster to be processed, the counterbore cluster to be processed, or the column cluster to be processed on the current processing layer;

Step D: finishing the processing if the current processing layer is a last layer, or otherwise, adjusting the focus of the laser to a next processing layer, and then return to step C.

The number of the above processing layer is one, two, three, or more.

What is claimed is:

1. A nasal prosthesis, comprising a nasal bridge element, the nasal bridge element comprising an attachment surface configured to be attached to a muscle tissue of a human nasal bridge and an outer surface configured to be attached to a skin tissue of the human nasal bridge; wherein, the nasal prosthesis further comprises a fixing structure arranged on the nasal bridge element, the fixing structure is arranged at the nasal bridge element and being one selected from the group consisting of a through-hole cluster and/or a column cluster, a through-hole cluster and a counterbore cluster, and a column cluster and a counterbore cluster, wherein a top surface of the column cluster forms all or a part of the attachment surface or the outer surface at a side where the column cluster is arranged.

2. The nasal prosthesis of claim 1, wherein the fixing structure is a through-hole array extending from the attachment surface of the nasal bridge element to the outer surface of the nasal bridge element;
or alternatively, the fixing structure is the through-hole array extending from the attachment surface of the nasal bridge element to the outer surface of the nasal bridge element and the counterbore array arranged at the attachment surface of the nasal bridge element, wherein the counterbore array and the through-hole array are spaced apart and alternated with one another.

3. The nasal prosthesis of claim 1, wherein the fixing structure is one selected from the group consisting of:
a column array arranged at the attachment surface of the nasal bridge element;
a column array arranged at the attachment surface of the nasal bridge element and the counterbore array arranged at the outer surface of the nasal bridge element; and
a first column array and a second column array respectively arranged at the attachment surface of the nasal bridge element and the outer surface of the nasal bridge element.

4. The nasal prosthesis of claim 3, wherein each first column of the first column array arranged at the attachment surface of the nasal bridge element has the same transverse dimension as each second column of the second column array arranged at the outer surface of the nasal bridge element, and a height of each first column is higher than a height of each second column; or
each first column of the first column array arranged at the attachment surface of the nasal bridge element has the same height as each second column of the second column array arranged at the outer surface of the nasal bridge element, and the transverse dimension of each first column is smaller than the transverse dimension of each second column; or
the height of each first column of the first column array arranged at the attachment surface of the nasal bridge element is higher than that of each second column of the second column array arranged at the outer surface of the nasal bridge element, and the transverse dimension of each first column is smaller than the transverse dimension of each second column.

5. The nasal prosthesis of claim 1, wherein the through-hole cluster and/or the column cluster, or the through-hole cluster and the counterbore cluster, or the column cluster and the counterbore cluster are distributed at the whole attachment surface or the whole outer surface of the nasal bridge element where the clusters are arranged;
or alternatively, the through-hole cluster and/or the column cluster, or the through-hole cluster and the counterbore clusters, or the column cluster and the counterbore cluster are provided in the form of spaced sections along a length of the nasal bridge element on the attachment surface or the outer surface of the nasal bridge element where the clusters are arranged.

6. The nasal prosthesis of claim 2, wherein the through-hole cluster and/or the column cluster, or the through-hole cluster and the counterbore cluster, or the column cluster and the counterbore cluster are distributed at the whole attachment surface or the whole outer surface of the nasal bridge element where the clusters are arranged;
or alternatively, the through-hole cluster and/or the column cluster, or the through-hole cluster and the counterbore clusters, or the column cluster and the counterbore cluster are provided in the form of spaced sections along a length of the nasal bridge element on the attachment surface or the outer surface of the nasal bridge element where the clusters are arranged.

7. The nasal prosthesis of claim 4, wherein the through-hole cluster and/or the column cluster, or the through-hole cluster and the counterbore cluster, or the column cluster and the counterbore cluster are distributed at the whole attachment surface or the whole outer surface of the nasal bridge element where the clusters are arranged;
or alternatively, the through-hole cluster and/or the column cluster, or the through-hole cluster and the counterbore clusters, or the column cluster and the counterbore cluster are provided in the form of spaced sections along a length of the nasal bridge element on the attachment surface or the outer surface of the nasal bridge element where the clusters are arranged.

8. The nasal prosthesis of claim 1, wherein the nasal bridge element is provided with a strip-shaped smooth region along an edge thereof;
the through-hole cluster and/or the column cluster, or the through-hole cluster and the counterbore cluster, or the column cluster and the counterbore cluster are arranged within a region enclosed by the smooth region on the attachment surface or the outer surface of the nasal bridge element where the clusters are arranged; and
the through-hole cluster and/or the column cluster, or the through-hole cluster and the counterbore cluster, or the column cluster and the counterbore cluster are provided in the form of spaced sections along a length of the nasal bridge element within the region enclosed by the smooth region on the attachment surface or the outer surface of the nasal bridge element where the clusters are arranged.

9. The nasal prosthesis of claim 2, wherein the nasal bridge element is provided with a strip-shaped smooth region along an edge thereof;
the through-hole cluster and/or the column cluster, or the through-hole cluster and the counterbore cluster, or the column cluster and the counterbore cluster are arranged within a region enclosed by the smooth region on the attachment surface or the outer surface of the nasal bridge element where the clusters are arranged; and
the through-hole cluster and/or the column cluster, or the through-hole cluster and the counterbore cluster, or the column cluster and the counterbore cluster are provided in the form of spaced sections along a length of the nasal bridge element within the region enclosed by the smooth region on the attachment surface or the outer surface of the nasal bridge element where the clusters are arranged.

10. The nasal prosthesis of claim 4, wherein the nasal bridge element is provided with a strip-shaped smooth region along an edge thereof;
the through-hole cluster and/or the column cluster, or the through-hole cluster and the counterbore cluster, or the column cluster and the counterbore cluster are arranged within a region enclosed by the smooth region on the attachment surface or the outer surface of the nasal bridge element where the clusters are arranged; and
the through-hole cluster and/or the column cluster, or the through-hole cluster and the counterbore cluster, or the column cluster and the counterbore cluster are provided in the form of spaced sections along a length of the nasal bridge element within the region enclosed by the smooth region on the attachment surface or the outer surface of the nasal bridge element where the clusters are arranged.

11. The nasal prosthesis of claim 5, further comprising a nasal tip element connected with the nasal bridge element; or further comprising the nasal tip element and a nasal columella element, wherein the nasal bridge element, the nasal tip element, and the nasal columella element are sequentially connected to form a shape of a human nose.

12. The nasal prosthesis of claim 6, further comprising a nasal tip element connected with the nasal bridge element; or further comprising the nasal tip element and a nasal columella element, wherein the nasal bridge element, the nasal tip element, and the nasal columella element are sequentially connected to form a shape of a human nose.

13. The nasal prosthesis of claim 7, further comprising a nasal tip element connected with the nasal bridge element; or further comprising the nasal tip element and a nasal columella element, wherein the nasal bridge element, the nasal tip element, and the nasal columella element are sequentially connected to form a shape of a human nose.

14. The nasal prosthesis of claim 8, further comprising a nasal tip element connected with the nasal bridge element; or further comprising the nasal tip element and a nasal columella element, wherein the nasal bridge element, the nasal tip element, and the nasal columella element are sequentially connected to form a shape of a human nose.

15. The nasal prosthesis of claim 9, further comprising a nasal tip element connected with the nasal bridge element; or further comprising the nasal tip element and a nasal columella element, wherein the nasal bridge element, the nasal tip element, and the nasal columella element are sequentially connected to form a shape of a human nose.

16. The nasal prosthesis of claim 10, further comprising a nasal tip element connected with the nasal bridge element; or further comprising the nasal tip element and a nasal columella element, wherein the nasal bridge element, the nasal tip element, and the nasal columella element are sequentially connected to form a shape of a human nose.

17. A method for manufacturing a nasal prosthesis, the nasal prosthesis comprising a nasal bridge element, and the nasal bridge element comprising an attachment surface configured to be attached to a muscle tissue of a human nasal bridge and an outer surface configured to be attached to a skin tissue of the human nasal bridge; wherein the method comprises: processing the nasal bridge element by a laser to form a fixing structure arranged on the nasal bridge element and being one selected from the group consisting of a through-hole cluster and/or a column cluster, a through-hole cluster and a counterbore cluster, and a column cluster and a counterbore cluster, and a top surface of the column cluster forms all or a part of the attachment surface or the outer surface at a side where the column cluster is arranged; and the processing by the laser comprises the following steps:
S1: dividing a region to be processed of the nasal bridge element of the nasal prosthesis into a processing layer;
S2: adjusting a focus of the laser on a processing layer of a through-hole cluster to be processed, a counterbore cluster to be processed, or a column cluster to be processed;
S3: performing processing at a position of the through-hole cluster to be processed, the counterbore cluster to be processed, or the column cluster to be processed on the current processing layer; and
S4: finishing the processing if the current processing layer is a last layer, or otherwise, adjusting the focus of the laser to a next processing layer, and then return to S3.

18. The method of claim 17, wherein the number of the processing layer is one, two, three, or more.
